(19) United States
(12) Patent Application Publication
(10) Pub. No.: US 2007/0093554 A1
(43) Pub. Date: Apr. 26, 2007

(54) AGENT FOR IMPROVING FEELING OF COLD

(76) Inventors: Miho Komatsu, Tsukuba-shi (JP); Koji Morishita, Tsukuba-shi (JP); Akemi Ogawa, Ichikawa-shi (JP); Goro Hori, Tsuchiura-shi (JP)

Correspondence Address: DARBY & DARBY P.C., P. O. BOX 5257, NEW YORK, NY, 10150-5257, US

(21) Appl. No.: 11/381,840

(22) Filed: May 5, 2006

(30) Foreign Application Priority Data
Oct. 25, 2005 (JP) 2005-309195

(51) Int. Cl. A61K 31/198

(52) U.S. Cl. 514/564

(57) ABSTRACT

It is desirable to provide a medicament or nutritional food to improve feeling of cold and to create fulfilling life for people who are suffering from feeling of cold. Specifically, one object of the present invention is to provide an, agent, a food and drink, or a food additive which can be used for improving feeling of cold. An agent, a food and drink, or a food additive for improving feeling of cold containing ornithine or a salt thereof as an active ingredient can be provided according to the present invention.

### BACKGROUND OF THE INVENTION

### Incorporation by Reference

The present application claims priority under 35 U.S.C. §119 to Japanese Patent Application No. 309195/2005 filed on Oct. 25, 2005. The content of the application is incorporated herein by reference in its entirety.

1. Field of the Invention

The present invention is related to an agent; a food and drink; and a food additive for improving feeling of cold which comprises ornithine or a salt thereof as an active ingredient.

2. Description of the Related Art

It is considered that more than half of women have feeling of cold. Normally human body reacts to prevent body heat being away by contracting capillary blood vessel on the body surface when they feel cold, but after a certain period of time, more blood is fed into the capillary blood vessel to adjust body temperature by preventing the temperature of body surface to become to low. However, blood vessel of a person feeling of cold is constricted as it is so that the person has feeling of cold in hands and feet. Further, even when the surrounding area becomes warm, the blood vessel does not open up for a while and it takes a longer time to recover.

One of the causes for feeling of cold is considered to be shortage of exercise, eating habit and a malfunction of adjustment system of autonomic nerve working in contraction and dilatation of blood vessel.

As a method for improving feeling of cold, bathing, exercise, improvement of eating habit, and massage are known.

Ornithine is being used, mostly in the U.S., as a food additive to strengthen muscle formation by letting the body secrete growth hormone or to prevent obesity by enhancing basal metabolism. Further, ornithine is used to produce L-ornithine-L-aspartate which is a medicament used to improve a liver disorder in Europe.

Arginine metabolically closely related to ornithine is reported to have a vasodilator activity and an activity to improve fluidity of blood. On the other hand, ornithine has no effect to compensate shortage of arginine (Journal of nutrition, 124(10), 1950-1960, 1994). Further, ornithine is known not to have a vasodilator action in human forearm (European Journal of Clinical Investigation, 26(4), 325-331, 1996) and a polyamine synthesized from ornithine as a precursor is negated to be responsible for fever (Journal of Pharmacy & Pharmacology, 37(5), 365-366, 1985).

### SUMMARY OF THE INVENTION

It is desirable to provide a medicament or nutritional food to improve feeling of cold and to create fulfilling life for people who are suffering from feeling of cold. Specifically, one object of the present invention is to provide an agent, a food and drink, or a food additive which can be used for improving feeling of cold.

The present invention relates to the following aspects (1) to (3):
(1) an agent for improving feeling of cold which comprises ornithine or a salt thereof as an active ingredient.
(2) a food and drink, or a food additive for improving feeling of cold which comprises ornithine or a salt thereof as an active ingredient.
(3) a method for improving feeling of cold which comprises administering or ingesting ornithine or a salt thereof to a subject in need thereof.

### BRIEF DESCRIPTION OF THE DRAWINGS

FIG. 1 is scale graphs expressing questionnaires for evaluation using Visual Analogue Scale (VAS) method. Each end of the segment has a criterion of expression.

FIG. 2 is a graph showing improvements in feeling of cold by the ingestion of ornithine. The vertical axis shows an average improvement ratio (%) of each criterion.

### DETAIL DESCRIPTION OF THE INVENTION

Ornithine as applied in the present invention includes L-ornithine and D-ornithine, preferably L-ornithine.

Ornithine can be obtained by a chemical synthetic method or a fermentation method. Also, ornithine is commercially available.

A chemical synthetic method can be found in, for example, Coll. Czechoslov. Chem. Commun., 24, 1993 (1959).

A fermentation method is disclosed, for example, in Japanese Published Unexamined Patent Application Nos. 24096/78 and 119194/86.

L-ornithine and D-ornithine can be also purchased from, for example, Sigma Aldrich Company.

Salts of ornithine include acid addition salts, metal salts, ammonium salts, organic amine addition salts, amino acid addition salts and the like.

The acid addition salts include inorganic acid salts such as hydrochloride, hydrosulfate, nitrate and phosphate; and organic acid salts such as acetate, maleate, fumarate, citrate, malate, lactate, α-ketoglutarate, gluconate and caprylate.

The metal salts include alkali metal salts such as sodium salt and potassium salt; alkaline earth metal salts such as magnesium salt and calcium salt; aluminum salt, zinc salt and the like.

Ammonium salts include salts of ammonium, tetramethylammonium and the like.

Organic amine addition salts include salts of morpholine, piperidine and the like.

Amino acid addition salts include salts of glycine, phenylalanine, lysine, aspartate, glutamate and the like.

Among the above salts of ornithine, hydrochloride, citrate, malate, α-ketoglutarate and aspartate are preferably applied, but one of the remaining salts or two or more of the above salts can be arbitrarily used.

In addition to ornithine and its salt, a proper additive for each application can be added to an agent, a food and drink, or a food additive of the present invention.

The above additive includes amino acids such as valine, leucine, isoleucine, arginine, lysine, glutamine, alanine, serine, glycine, cystein and threonine, and the like.

Administration or ingestion of; an agent; a food and drink; or a food additive for improving of feeling of cold; can improve feeling of cold.

Feeling of cold, in which hands and feet are cold, and waist is cold, can be improved in coldness at hands, feet and waist by administration or ingestion of the agent; the food and drink; or the food additive for improving of feeling of cold according to the present invention.

An agent according to the present invention comprises ornithine or a salt thereof, and may include one or more pharmaceutically acceptable carriers, as necessary, and another active ingredient for other medical treatments as necessary.

An agent according to the present invention can be produced by mixing ornithine or a salt thereof with carriers and the like as necessary, according to an arbitrary method well known in the technological field of pharmaceuticals.

Aa an administration route, it is preferable to use the most effective route for treatment. Oral route and parenteral route such as intravenous administration and the like can be used, but oral route is preferable.

Dosage forms include a tablet, powder, granules, syrup and injections.

Liquid preparation such as syrup which is suitable for an oral administration can be prepared of water; sugars such as sucrose, sorbitol, and fructose; glycols such as polyethylene glycol and propylene glycol; oils such as sesame oil, olive oil and soybean oil; preservatives such as p-hydroxybenzoic acid ester; flavors such as strawberry flavor and peppermint and the like.

In addition, a tablet, powder, granules and the like can be prepared of excipients such as lactose, glucose, saccharide and mannitol; disintegrants such as starch and sodium alginate, lubricants such as magnesium stearate and talc, binders such as polyvinyl alcohol, hydroxypropyl cellulose and gelatin; surfactants such as fatty acid ester; plasticizers such as glycerin and the like.

The formulation suitable for parenteral administration preferably comprises a sterilized aqueous preparation containing ornithine or a salt thereof which is isotonic to the recipient's blood. For example, for injections, an injectable solution may be prepared by using a carrier such as a salt solution, a glucose solution, and a mixture of a salt solution and a glucose solution.

In these parenteral preparations, it is possible to add one or more of supplementary components for the oral preparations mentioned above, selected from diluents, preservatives, flavors, excipients, disintegrants, lubricants, binders, surfactants, plasticizers and the like.

In case of an orally administered agent according to the present invention, although the concentration of ornithine or a salt thereof can be properly selected in accordance with a kind of the orally administered agent and an effect expected by administration of the orally administered agent, it is normally in a range of 0.1 to 90% by weight of ornithine or a salt thereof; preferably in a range of 0.5 to 80% by weight, and most preferably in a range of 1 to 70% by weight.

Although a dosage of the agent according to the present invention may vary depending on such factors as an administration form, and age and body weight of the person who is being administered to, in case of an orally administered agent, for an adult per day, it is normally in a range of 50 mg to 30 g of ornithine and a salt thereof, preferably in a range of 100 mg to 10 g, and most preferably in a range of 200 mg to 3 g, which is administered one time or separately a few times. Although a period of administration is not limited, it is normally in a range of one day to one year, preferably in a range of one week to three months.

The food additive according 'to the present invention can be prepared by the same method applied for the above agent. The food additive is normally mixed or resolved with another food additive as necessary and processed into, for example, powders, granules, a pellet, a tablet or various solutions.

As the food or drink according to the present invention, mention may be made of the food or drink comprising ornithine or a salt thereof, or a food additive according to the present invention.

The food or drink according to the present invention can be processed and manufactured by a general food and drink manufacturing method except that ornithine or a salt thereof, or the food additive according to the present invention is added to the known food or drink.

The food or drink according-to the present invention can be manufactured by granulation methods such as a fluidized-bed granulation, a stirring granulation, an extrusion granulation, a rolling granulation, an airstream granulation, a compression molding granulation, a disruption granulation, a spray granulation or a blasting granulation; coating methods such as a pan coating, a fluidized-bed coating and a dry coating; a plumping method such as a puff drying, an excess steam method, a foam mat method or a microwave heating method; or an extrusion method using, for example, an extruding granulator or an extruder.

The food or drink according to the present invention includes juices; soft drinks; teas, dairy products such as lactic acid bacteria beverages, fermented milk, frozen dessert, butter, cheese, yogurt, processed milk and defatted milk; animal meat products such as ham, sausage and hamburger; fish cake products such as plate-like fish cake or kamaboko in Japanese, pipe-like fish cake or chikuwa in Japanese, and fried fish cake or satsumaage in Japanese; egg products such as rolled egg with soup or dashimaki in Japanese and egg-tofu; confectioneries such as cookie, jelly, chewing gum, candy and snack; breads; noodles; pickles; smoked food products; dried fishes; fishes boiled in soy sauce or tsukudani in Japanese; salt curing products; soups; condiments; or any other forms.

Further the food or drink of the present invention may take the forms of powdery foods; sheet-like foods; bottled foods; canned foods; retort foods; capsule foods; tablet-like foods; fluid foods; nutritious supplement drinks or the like.

The food or drink according to the present invention can be used as health foods; functional foods; nutritious supplement foods; or food for specified health use, for improving feeling of cold.

A food additive such as a sweetener, a coloring agent, a preservative, a thickening stabilizer, an antioxidant, a color developing agent, a bleaching agent, a fungicide, a gum base, a bitter agent, an enzyme, a wax, a sour agent, a seasoning, an emulsifier, a nutrient supplement, an additional materials for preparation, a flavor and a spice extract, which are generally used in a food and drink, can be added to the food or drink or the food additive according to the present invention.

Although the concentration of ornithine or a salt thereof can be properly selected in accordance with a kind of a food or drink, and an effect expected by administration of the food or drink, it is normally in a range of 0.1 to 90% by weight of ornithine or a salt thereof; preferably in a range of 0.5 to 80% by weight, and most preferably in a range of 1 to 70% by weight.

Although an intake of the food or drink according to the present invention may vary depending on an ingestion form, and age and body weight of the person being ingested to, for an adult per day, it is normally in a range of 50 mg to 30 g of ornithine or a salt thereof, preferably in a range of 100 mg to 10 g, and most preferably in a range of 200 mg to 3 g, which is ingested one time or separately a few times. Although a period of ingestion is not specified, it is normally in a range of one day to one year, preferably in a range of one week to three months.

The followings are test examples of the effect of ornithine on improving feeling of cold.

### TEST EXAMPLE

Six tablets of example 1 (the tablet containing ornithine) or 6 tablets of comparative example 1 (the tablet not containing ornithine) were ingested to two groups with 7 subjects out of 14 normal subjects of each male and female between 45 to 64 years of age a day for 3 weeks.

Before the ingestion and just after the ingestion, the improvements in feeling of cold of each subject were evaluated using Visual Analogue Scale (VAS) method.

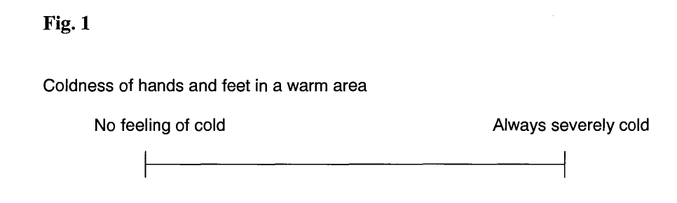

Specifically each end of the segment has a criterion of expression. Referring to FIG. 1, each subject marked somewhere in the line, corresponding to each term of the questionnaires. The distance (mm) from the left end of the line to the marked point was measured and the difference between before and after the test was calculated. The difference by the value before the test was shown in the percentage calculated; and each average value and standard deviation for each group were calculated. Further the average improvement ratio (%) was the value obtained by subtracting the average value of the placebo group from the average value of ornithine group. Further, it was confirmed that there was no difference between two groups before the administration test.

Further the test was carried out under a random assignment and the comparison between the double blind parallel groups was carried out. The test of the statistically significant difference between two groups was an unpaired t-test of both side distributions using the difference between the beginning and just after the test.

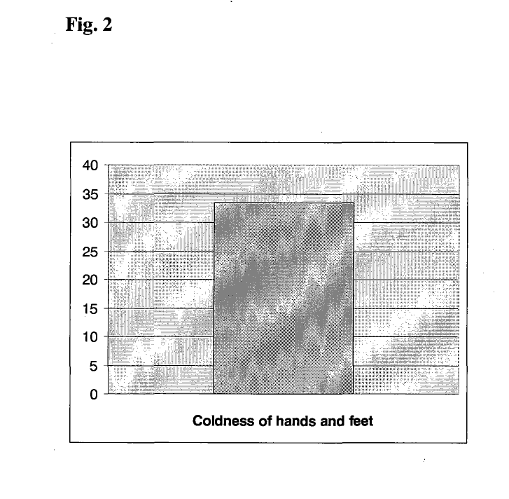

The results are shown in FIG. 2. According to ingestion of ornithine, coldness of hands and feet were improved remarkably. The improvement effect of ornithine on feeling of cold was demonstrated by this result.

The followings are the example of the present invention.

### Example 1

### Production of a Tablet containing Ornithine

A mixture of 136.2 Kg of ornithine hydrochloride (Commercial name: L-ornithine hydrochloride, Kyowa Hakko Kogyo Co., Ltd.); 36.0 Kg of a fine cellulose crystal (Commercial name: Avicel FD101, Asahi Kasei Chemicals Co., Ltd.); 6.6 Kg of sucrose fatty acid ester (Commercial name: DK ester F-20W, Daiichi Kogyo Seiyaku Co. Ltd.); 1.2 Kg of calcium phosphate (Commercial name: Tricalcium phosphate, Taihei Chemical Industrial Co., Ltd.); and 20.0 Kg of β-cyclodextrin (Commercial name: Seldex B-100, Nihon Shokuhin Kako Co., Ltd.); was mixed using a conical blender (CB-1200 Blender, Nihon Kansoki Co., Ltd.). The mixture obtained was compressed and molded to a tablet of 250 mg with 8 mm of diameter under 10 KN of compression-molding pressure using the rotary compression molding machine (VIRG0524SS1AY, Kikusui Seisakusyo Ltd.).

### Example 2

### Production of an Enteric Capsule containing Ornithine

A mixture of 20 Kg of the mixture prepared in Example 1 and 0.2 Kg of silicon dioxide was mixed and stirred. The mixture obtained was put into a capsule-filling machine to fill 20,000 tablets of gelatin Number 2 hard-capsules to provide the hard-capsules. The surfaces of the hard-capsules obtained were coated with a zein solution using High Coater HCT-48 (Freund Corporation) to produce 20,000 enteric capsules containing ornithine hydrochloride.

### Example 3

### Production of Enteric Tablet containing Ornithine

The surfaces of the tablets prepared in Example 1 were coated with shellac solution using High Coater HCT-48 (Freund Corporation) to produce an enteric tablet.

### Example 4

### Production of a Drink containing Ornithine

Each 1.28 Kg of ornithine hydrochloride (Commercial name: L-ornithine hydrochloride, Kyowa Hakko Kogyo Co. Ltd.); 3 Kg of erythritol (Nikken Kagaku Co. Ltd.); 0.05 Kg of citric acid (Kyowa Hi Foods Co. Ltd.); 3 g of artificial sweetener; and 0.06 g of flavor were stirred and dissolved in 50 L of water at solution temperature 70° C.; After the pH of the solution was adjusted to 3.3, the solution was sterilized using plate sterilization and filled into bottles. The bottle was sterilized using a pasteurizer to produce the ornithine beverage.

### Comparative Example 1

Instead of ornithine hydrochloride in Example 1, the same amount of lactose was used to produce a tablet not containing ornithine.

While the invention has been described in detail and with reference to specific embodiments thereof, it will be apparent to one skill in the art that various changes and modifications can be made therein without departing from the spirit and scope thereof. All references cited herein are incorporation in their entirety.

What is claimed is:

1. An agent for improving feeling of cold which comprises ornithine or a salt thereof as an active ingredient.

2. A food and drink, or a food additive for improving feeling of cold which comprises ornithine or a salt thereof as an active ingredient.

3. A method for improving feeling of cold which comprises administering or ingesting ornithine or a salt thereof to a subject in need thereof.
